(19) United States
(12) Patent Application Publication
(10) Pub. No.: US 2024/0307571 A1
(43) Pub. Date: Sep. 19, 2024

(54) DRYING AND CLEANSING APPARATUS

(71) Applicant: Jonathan Traub, San Diego, CA (US)

(72) Inventor: Jonathan Traub, San Diego, CA (US)

(21) Appl. No.: 18/572,756

(22) PCT Filed: Jun. 21, 2022
(86) PCT No.: PCT/IB2022/055741

§ 371 Date: Dec. 20, 2023

(30) Foreign Application Priority Data
Jun. 23, 2021 (ZA) 2021/04305

(51) Int. Cl. A61L 2/10; A61L 2/26; F26B 3/04

(52) U.S. Cl. CPC A61L 2/10; A61L 2/26; F26B 3/04; A61L 2202/11; A61L 2202/14; A61L 2202/23

(57) ABSTRACT

Apparatus for treating an object which has a housing into which hot air and ultraviolet radiation are directed to impinge on the object which is located inside the housing, if the object is a container, inside the housing, the hot air and UV radiation are directed into an interior of the container.

### BACKGROUND OF THE INVENTION

This invention relates to an apparatus for treating an object.

As used herein “treating” includes at least one of the following: drying, warming, and sterilising, an object.

The object which is treated may be a single object or may be one of a number of similar or different objects. The object may comprise a container such as a bottle, a jar, a utensil or any other device which is used for containing or transporting foodstuffs and which is to be made available for use in a clean, sanitised and hygienically acceptable manner. Other objects given only by way of non-limiting examples, include keys, cell phones and cell phone covers.

More generally the apparatus of the invention is suited for the treating of an object of any nature which is correctly sized for use with the apparatus so that the object can be dried and sterilised according to requirement.

Typically, when a reusable container or bottle which contains, say, milk for an infant, is emptied the bottle is cleaned by washing and, after the bottle has been dried, it is replenished. A conventional bottle can readily be washed but, if the bottle has a narrow neck or mouth, it is not easy to dry an interior of the bottle effectively. If the bottle is returned to a storage location, probably with a sealing cap engaged with its mouth, then moisture which may inadvertently be retained inside the bottle can encourage the growth of mould and lead to an interior of the bottle being contaminated. A typical consumer reaction to this situation is to discard the bottle so as to avoid dealing with the problem.

Merely by way of non-limiting examples other objects which should be thoroughly cleaned for hygienic purposes and to avoid contagious effects include baby pacifiers, keys, toothbrushes, cellular phones and phone covers, and the like.

The invention is not limited as to the nature of the object, or objects, which can be treated.

An object of the present invention is to provide an apparatus which, to a substantial extent, allows for the aforementioned issues to be addressed for a range of diverse objects.

### SUMMARY OF THE INVENTION

The invention provides apparatus for treating an object, which apparatus comprises a housing which is impervious to ultraviolet radiation and which includes a volume within which the object is removably located, a mechanism which directs a stream of conditioned air into the volume, and an ultraviolet source positioned to direct ultraviolet radiation into the volume.

The mechanism may include an air heater, an air fan, and a controller for regulating operation of the air heater and the air fan thereby to produce said stream of conditioned air at a predetermined air temperature.

The controller may be operable to produce said stream of conditioned air at a predetermined air flow rate.

In one embodiment the apparatus includes a base in which a passage is located, a vent inlet to the passage and an outlet from the passage, said air heater and air fan being positioned in the passage, the base including a platform at which said outlet from the passage is positioned, said housing including an inlet and being configured to be engaged with the platform whereby said outlet from the passage is placed in air communication with said inlet to said housing.

A water outlet from the passage may be located at what, in use, is a lowermost point of the passage.

Preferably, in use, said vent inlet to the passage is higher than said air fan and said air heater. Other arrangements are however possible.

The ultraviolet source, which is operable by the controller, may be located at said outlet from the passage.

The apparatus may include a safety switch at the platform which prevents operation at least of the ultraviolet source unless the housing is engaged with the platform.

The platform may be configured to support a container which is inside said volume and which has a mouth which is positioned over said outlet from the passage.

To limit the effect of a possible malfunction the apparatus may include a fuse which, in response to a temperature of said stream of conditioned air above a threshold value, inhibits further operation of said mechanism.

The apparatus may include a display which provides information relating to the operation of the apparatus. The display may for example indicate the duration or period for which the apparatus is operable. Another possibility is for the display to indicate the temperature of the air and of the air flow rate.

The ultraviolet source may include a single source or a plurality of sources. Typically the source comprises one or more light-emitting diodes which function at a desired frequency. The radiation which is emitted by the source is at a suitable wavelength in order to sterilise and sanitise the object or objects which are inside the housing.

The apparatus may be powered by means of a mains supply but it is also possible to make use of an onboard battery which is rechargeable from a mains supply to power the apparatus. If the display is used then the display may indicate the status of the battery i.e. the degree of charge in the battery.

An advantage of using an onboard system for powering the apparatus is that the portability of the apparatus is not affected. The apparatus can also be used, with safety, in damp environments e.g. next to a washing location.

The controller may produce a signal to indicate that a particular treatment process has been completed. The signal may be visual or audible or both.

In another specific embodiment the invention provides an apparatus for treating an object, the apparatus including a housing with a wall, which is impermeable to ultraviolet (UV) radiation, surrounding a volume and an inlet into the volume, a base upon which the housing can be positioned with its inlet facing downwardly, a member which is mounted to the base and which is configured to extend into the volume through the inlet, a UV source which emits UV radiation at a suitable wavelength into the volume in order to sterilise and sanitise at least a part of an object in the volume, and a heater and fan arrangement which is operable to direct heated air into the volume in order to dry at least a part of the object.

The member may be detachably mounted to the base.

The member may be tubular or it may support a tube. Air from the fan may pass through the tubular member or the tube into the volume.

The member may include one or more outlets through which air is released into the volume.

Preferably the member is elongate and tubular and has a plurality of outlets at spaced locations along its length which are positioned to direct air from inside the tubular member into the volume.

Air from the elongate member may be directed radially outwardly. If the object, to be treated, is a container with a mouth then the container may be positioned on the base, inside the housing, with the mouth of the container facing downwardly and with the member extending into the interior of the container. The air may then impinge effectively on an entire inner surface of a wall of the container.

The UV source may comprise a single source or a plurality of sources. Typically, the UV source comprises a plurality of UV sources such as light emitting diodes which function at a desired frequency.

The UV source may be configured so that an entire inner surface of the wall of the container is subjected to UV radiation.

Alternatively or additionally a source of UV radiation may be positioned on the base to direct UV radiation into the volume.

The UV source may be operable continuously or it may be operable in a pulse mode.

The apparatus may include a display which provides information relating to the operation of the apparatus. The display may for example indicate the duration of a timing period. Another possibility is for the display to indicate the status of a power source which is used in the operation of the apparatus.

Features of any other embodiment, described herein, may be used with this specific embodiment, and vice versa.

The apparatus may include a safety switch which ensures that the apparatus is operable only when the housing is engaged with the base. In the absence of the housing the UV source cannot be energised.

Air which is directed into the volume may be drawn through one or more inlets which may be provided in the base.

The apparatus may be designed to rest on a tray or the like which collects moisture which may, despite the drying process effected by the air fan, drain from the base.

To allow for the apparatus to be used with objects of different sizes the duration of the drying period may be adjustable.

The member by means of which air is directed into the volume may be detachably engaged with the base to facilitate package, storage and cleaning purposes. If the UV sources are mounted to the member, then the UV sources may be mounted to the member at spaced apart locations.

It is also possible to provide the apparatus in the form of a kit comprising a base of the aforementioned kind and a plurality of the members each of which is configured to be used in an effective manner with a housing, or a container, of a respective size. This is particularly useful in commercial settings where a number of different size containers are to be sanitised successively.

### BRIEF DESCRIPTION OF DRAWINGS

The invention is further described by way of examples with reference to the accompanying drawings in which:

FIG. 1 is a front perspective view of apparatus according to the invention,

FIG. 2 is a front view of the apparatus of FIG. 1,

FIG. 3 is a simplified side view in section of the apparatus shown in FIG. 2, taken on a line 3-3 and illustrating, in addition, a container which is undergoing treatment, and schematically representing some electrical components used in the apparatus,

FIG. 4 is a plan view from below of the apparatus of FIG. 1,

FIG. 5 shows on an enlarged scale a nozzle accessory, and also how the nozzle accessory is used,

FIG. 6 illustrates the apparatus, in use, with a container with a narrow neck, and a support for the container,

FIG. 7 depicts a support which is positioned inside a housing of the apparatus,

FIGS. 8 and 9 show how the support of FIG. 7 can be used to support a number of different objects which are to be treated,

FIG. 10 is a side view of apparatus according to another embodiment of the invention, and

FIG. 11 shows in section the apparatus of FIG. 10 in use.

### DESCRIPTION OF PREFERRED EMBODIMENTS

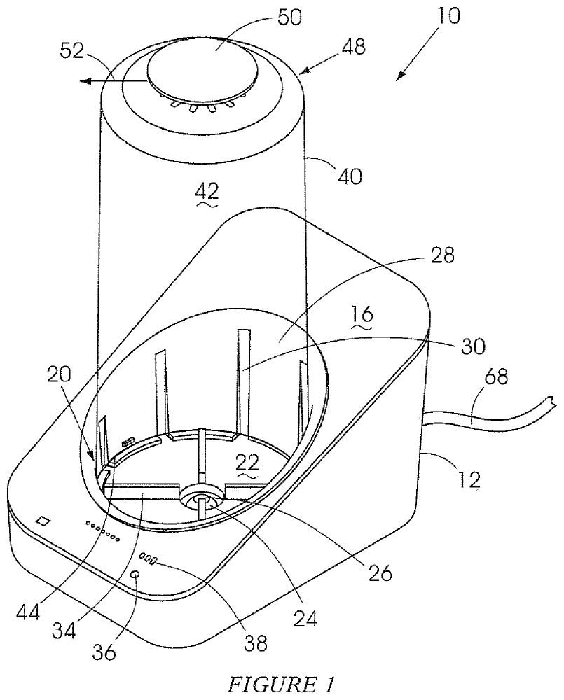

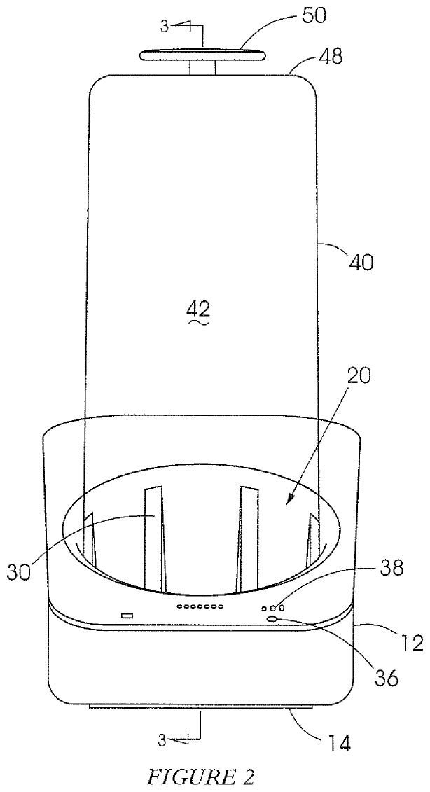

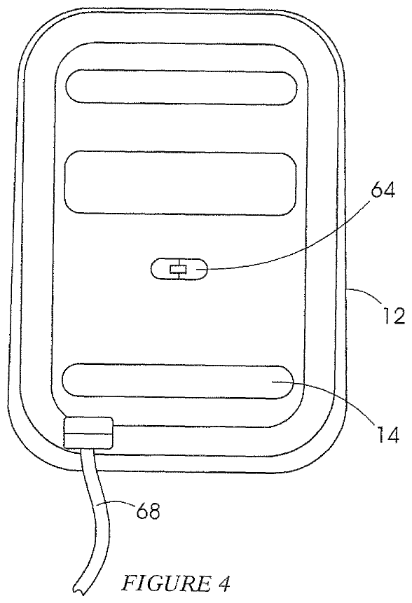

FIGS. 1, 2, 3 and 4 of the accompanying drawings are different view of apparatus 10 according to the invention.

The apparatus 10 includes a base 12 which is mounted on rubber feet 14 which could, possibly, be in the form of suction pads. On an upper surface 16 the base has a recess 20. A lower surface of the recess defines a platform 22. Centrally positioned in the platform 22 is an air outlet 24. A light emitting diode comprising an ultraviolet (UV) source 26 is positioned in the outlet 24 facing upwardly. A wall 28 of the recess 20 is formed with a number of spaced apart air escape grooves 30.

Flexible support ribs 34 radiating from the outlet 24 stand on the platform 22.

Capacitive touch buttons 36 on a lower part of the upper surface 16 allow for control functions to be exercised over the apparatus 10 as is described hereinafter. Indicators 38 e.g. LEDs provide visual and audible information, relating to the operation of the apparatus, in use. If desired use can be made of sound generators to provide similar operational information. Capacitive touch buttons are preferred in that no gaps are formed between the buttons and the surrounding surface 16 which could allow for the ingress of water or dust. Nonetheless physical knobs or buttons could be used in place of the capacitive buttons.

A housing 40 of circular cylindrical form is removably engaged with the recess 20. The housing 40 encloses a volume 42 and has a downwardly facing inlet 44 which fits inside the recess 20 resting on the ribs 34. The air escape grooves 30 are in air communication with small gaps between the inlet 44 to the volume 42 and the surface of the platform 22 due to the spacing effect of the ribs 34.

At an upper end 48 the housing has a gripping formation 50. Air escape vents 52 are formed between the gripping formation and a surface of the upper end 48.

The housing 40 and the gripping formation 50 are made from a material which is not permeable to radiation from the ultraviolet source 20, and thus act as a blocking filter for UV radiation.

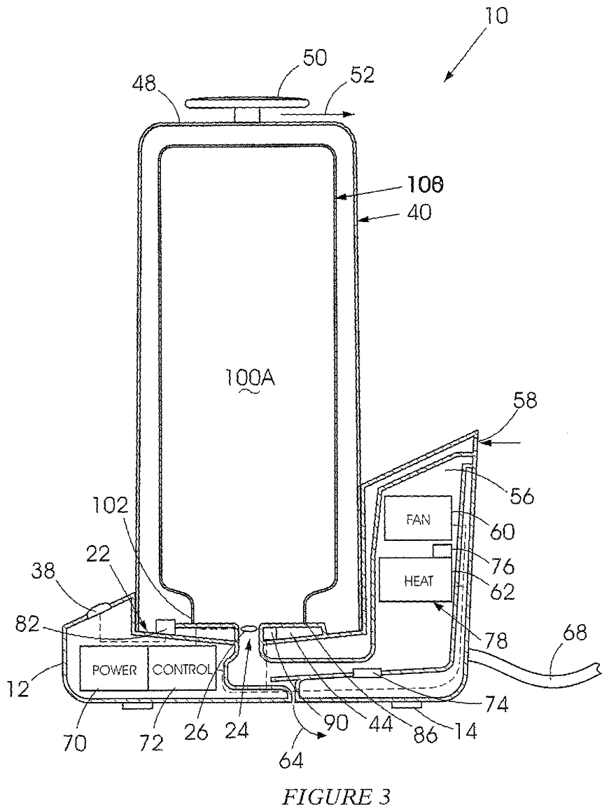

Referring to FIG. 3 an air passage 56 inside the base 12 extends from an air inlet vent 58 to the air outlet 24 in the platform 22. An air fan 60 is located inside the passage 56 adjacent but lower than the air vent 58. An air heater 62 in the passage is adjacent the air fan 60. The passage has a water outlet 64, which is located at what, in use, is a lowermost portion of the passage-as illustrated in FIG. 3. This ensures that water which may accumulate inside the passage is drained, automatically, from the passage.

FIG. 3 schematically depicts electrical components, including the air fan 60 and the heater 62, of the apparatus 10. The apparatus is supplied with electrical power via a mains supply cable 68, shown in FIG. 1. A transformer, rectifier and a DC conditioning circuit 70 produce a DC voltage at a desired level from the mains supply to operate the various components. The electrical components include a microprocessor controller 72 which is operable, at least, in response to the touch buttons 36 shown in FIG. 1.

The fan 60 is a low-voltage DC fan positioned to draw in air through the air inlet vent 58 which is designed and positioned so that water cannot easily enter the passage 56 if the base 12 is stationary on a horizontal surface. In use the fan 60 blows air through the heater 62 which is a high voltage internal ceramic heating element. The heated air flows along the passage 56 to the air outlet 24.

Operation of the air heater 62 is regulated via control signals from the controller 72. The maximum air temperature is regulated by a resettable bimetallic thermostat 74 with a fixed set point. When the air temperature exceeds the set point, power to the air heater 62 is disconnected irrespective of the state of the control signal from the controller 72. When the bimetallic thermostat 74 cools below its reset temperature, power is again connected to the air heater 62.

The duty cycle of the air heater 62 can be varied by the controller 72 in order to regulate the temperature of the air in the passage. Additionally, the controller 72 can vary the operational speed of the DC fan 60 via control signals. Thus the rate of air flow through the air heater 62 and hence through the passage 56 can be adjusted. By varying the air flow rate the temperature of the flow of air can also be controlled.

A thermal cut-off fuse 76 is mounted on a casing 78 of the heater 62 and is connected in series with the power supply to the heater. This is a non-resettable, normally closed, safety fuse. If the temperature of the casing 78 to which the thermal cut-off fuse is exposed, exceeds the trip temperature of the cut-off fuse 76, then the cut-off fuse opens and power to the air heater 62 is permanently disabled.

In use the ultraviolet source 26 produces UV radiation at a germicidal wavelength. The source 26 is located in the outlet 24 from which hot air exits the passage 56 and radiates a cone of UV light upwardly and outwardly from the air outlet 24 into the volume 42. The controller 72 can vary the current powering the ultraviolet source 26 and also the time period for which the ultraviolet source 26 is operable in order to optimise sterilisation times for various pathogens.

A safety switch 82 is positioned on the platform 22. This is a normally open switch that is only closed if the housing 40, which acts as a UV safety cover, is correctly positioned on the platform 22. The operation of the switch 82 is constantly monitored by the controller 72. The supply of power to the UV source 26 is only enabled when the switch 82 is closed. The power supply for the UV source 26 is immediately interrupted if the housing 40 is lifted from the platform 22 for the safety switch 82 is then automatically and immediately opened.

The UV LED source 26 is mounted on an aluminium substrate 86 which helps to cool the UV source 26. Air from the air fan 60 exiting from the outlet 24 passes over the UV source 26 and also helps to limit the operating temperature of the UV source.

A temperature sensor 90, positioned near the UV source 26, and mounted to the substrate 86 provides a measure of the air temperature at the air outlet 24. The sensor 90 is constantly monitored by the controller 72 and power to the UV source 26 is adjusted as needed to ensure that the operating temperature of the UV source is optimised for maximum effectiveness, and longevity, of the source.

FIG. 3 shows in cross section a container 100 with a mouth 102 which is positioned on the platform 22 so that heated air is blown directly from the passage 56 through the air outlet 24 into an interior 100A of the container 100. At the same time the interior 100A is subjected to the ultraviolet radiation. Thus the container interior is dried by the passage of the heated air and simultaneously sterilised.

Normally the container 100 is previously washed by a person in an appropriate manner. After the container has been rinsed it is placed, inverted as shown in FIG. 3, resting on the platform 22. The housing 40 is positioned over the container 100 and the safety switch 82 is thereby automatically closed. A user can then activate the apparatus 10 by means of control signals input via the buttons 36. The air heater 62 is energised and the air fan 60 is simultaneously activated. The UV source 26 is also energised. Heated and dried air is forced into the container 100A. The heated and dried air impinges on an inner surface 106 of the wall of the container 100. Air inside the container is pressurised to some extent but some air does escape through the mouth 102 of the container and then leaves the apparatus 10 either through the air escape grooves 30, or the vents 52. Radiation from the UV source 26 is directed onto the inner surface 100. This sanitises and leaves the surface in a hygienically acceptable state.

The aforementioned treatment process, comprising heating, drying and radiation, continues for a period determined by a timer embodied in the controller 72. The duration of the treatment process can be set by a user according to requirement. For a small container typically the period might be of the order of 60 to 90 seconds. In a number of other applications the period is meaningfully longer. At the end of this treatment period the controller 72 causes a signal to be displayed on indicators 38 and, at that time, the fan 60, the heater 62 and the UV source 26 are no longer energised.

At any time if the housing 40 is removed the safety switch 82 immediately and automatically interrupts operation of the UV source 26.

At the end of the treatment process the container can be removed from the apparatus and is then available for reuse. This is with the assurance that the container has been sterilised and is in a hygienically acceptable condition.

The indicators 38 may comprise a series of light emitting guides or other displays which according to their illumination indicate the extent to which the treatment process has been completed. The duration of a treatment process can be adjusted by a user using selected touch buttons 36 on the base.

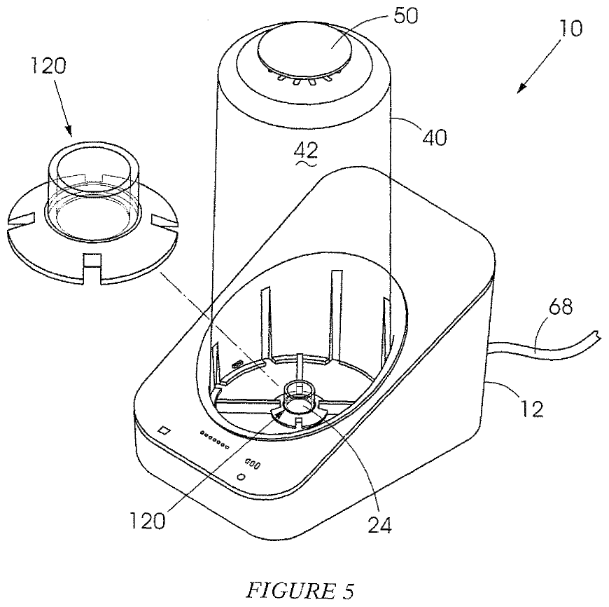

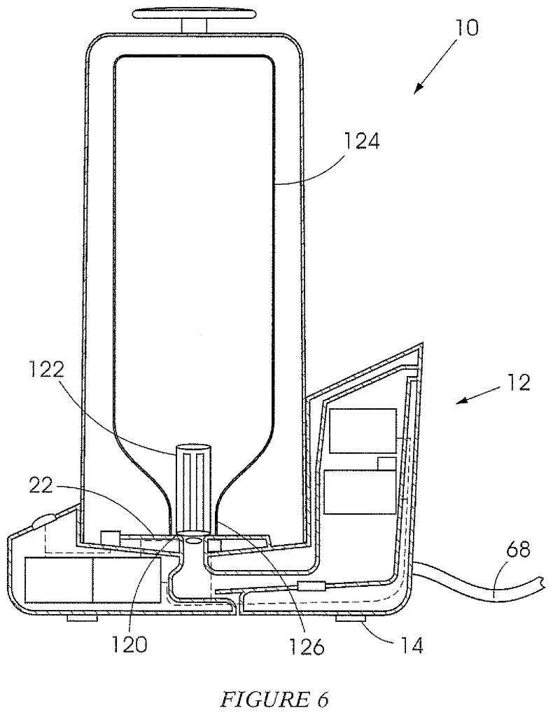

FIG. 5 illustrates on an enlarged scale a nozzle accessory 120 which can be positioned over the air outlet 24 in the platform 22 in order to direct the heated stream of air higher upwardly into the volume 42. The nozzle accessory 120 can be provided with upwardly extending arms 122 which fit inside a container 124 with a narrow mouth 126—see FIG. 6. The container 124 is treated in a similar manner to what has been described in connection with FIG. 3 but the arms 122 help to stabilise and centre the container 124 on the platform 22.

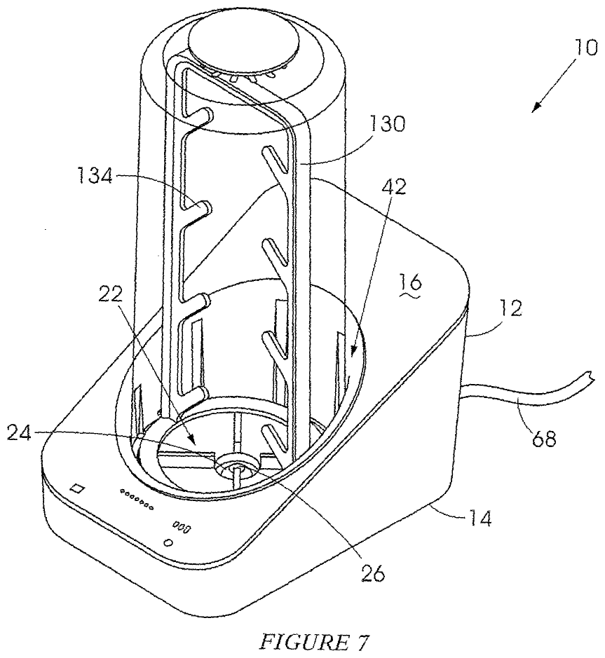

The apparatus of the invention can be used effectively for treating objects other than containers. FIG. 7 shows a support 130 which is located on the platform 22 positioned inside the volume 42. The support 130 has a number of hook formations 134 to which objects which are to be treated are attachable.

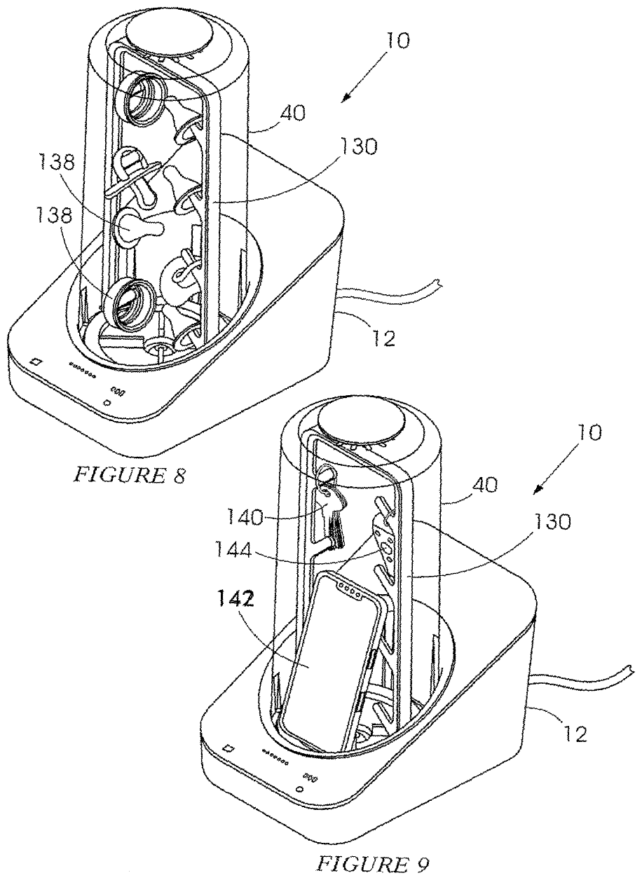

FIG. 8 shows, by way of example, the support 130 located on the base 12 and engaged with different objects 138 taken from a baby's bottle. FIG. 9 shows a set of keys 140, a cellular phone 142 and a whistle 144 attached to the support 130. The objects shown in FIG. 8 are typically washed beforehand and are then subjected to a drying and sterilisation process using the apparatus. With the FIG. 9 example the objects are probably not washed but nonetheless are sterilised using the heated air and the ultraviolet radiation.

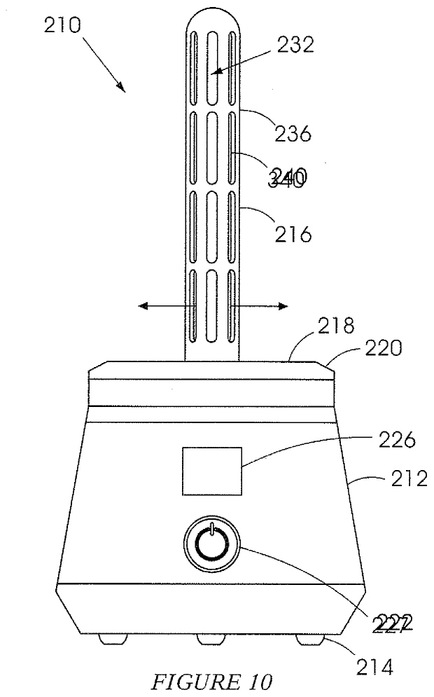

FIG. 10 is a side view of apparatus 210 according to another embodiment of the invention.

The apparatus 210 includes a base 212 which is mounted on rubber feet 214 and an elongate member 216 which extends upwardly from a platform 218 on an upper surface 220 of the base. A user-actuable control switch 222 is mounted to the base. This is below a display 226 which is used, as is described hereinafter, to provide visual information on operational aspects of the apparatus.

The elongate member 216 is tubular and includes an inner bore 232. The member 216 has a wall 236 through which are formed a plurality of elongate slots 240 positioned at spaced apart locations along a length of the member. The slots 240 constitute outlets from the bore 232.

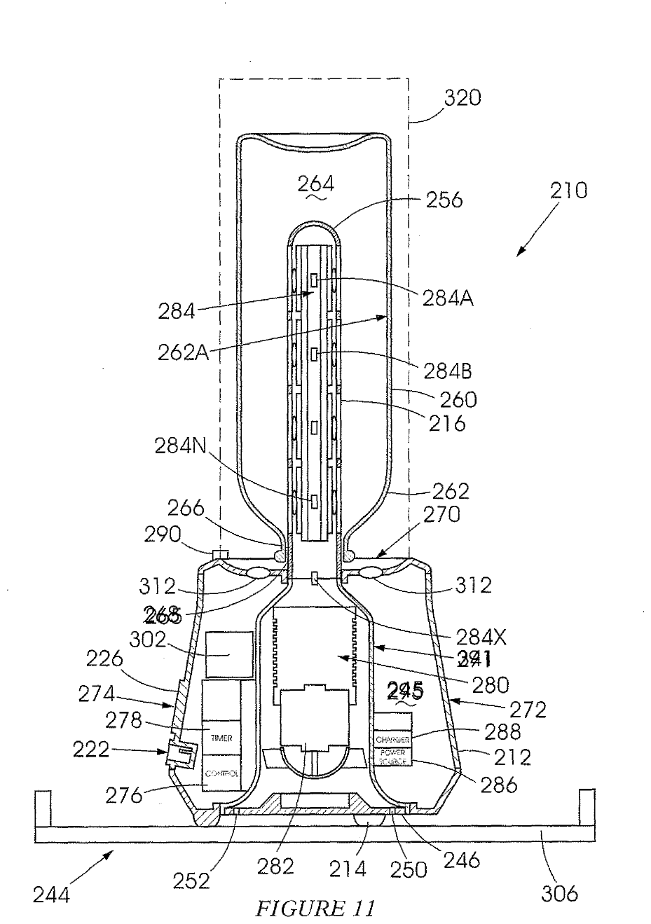

FIG. 11 shows the apparatus of FIG. 10 from one side and in cross section, in use.

A lower end 244 at the base 212 includes a plate 246 which has a plurality of perforations 250 and 252. The perforations 250 are designed to allow for air to flow into the interior of the base. The perforations 252 on the other hand are outlets to allow water to drain from the base, as is hereinafter described.

An upper end of the member 216 is sealed with a domed component 256.

An inverted container 260 is engaged with the apparatus (in use). The container has a wall 262 which encloses a volume 264 and a narrow neck 266 which terminates in a mouth 268 which rests on a platform 270. The container is made for example from glass or a plastics material.

The aforementioned functional aspects of the container 260 are given by way of example only.

Typically, the container is a multiuse container functioning as a receptacle for water, fruit juice or the like. Again, it is noted that these aspects are exemplary only for the use of the apparatus of the invention is not restricted to food or liquid containers.

The base 212 comprises an outer tubular structure 272 which is suitably designed to contain operative components 274 which are similar to those described in connection with FIG. 3. These components include a controller 276 which preferably is microprocessor based, a timer 278, a heater coil 280, an air fan 282, a UV source 284 which typically is in the form of a plurality of light emitting diodes which function at a suitable ultraviolet frequency, the display 226, a power source 286, an optional charging arrangement 288 and the switch 222.

The heater coil 280 is adjacent the fan 282. When the fan is operated air is drawn into the housing through the corresponding air inlets 250 in the plate 246. The heater coil 280 and the fan 282 are contained in an inner tubular structure 291. A lower end of the tubular structure is connected to the plate 246 so that air passing through the inlets 250 enters the inner tubular structure 291.

The power source 286 comprises a plurality of batteries 293 which are positioned in a space 295 between the outer tubular structure 272 and the inner tubular structure 291. The batteries may be replaceable or can be recharged by means of the charging arrangement 288 which comprises a suitable transformer and rectifying circuit connected to a plug (not shown), which may be a USB port, so that the batteries can be recharged, when desired, from a main supply. Alternatively a main supply (not shown) is used in place of the battery-based supply/USB arrangement.

Other elements of the components 274, collectively designated 302, are mounted inside the outer tubular structure 272 at appropriate locations.

FIG. 11 illustrates a tray 306 upon which the base 212 can rest with the rubber feet 214 in contact with an upper surface of the tray so that water which may drain is directed onto the tray 306 through one or more of the outlets 252.

The platform 270 has a number of openings 312 which are in communication with a number of tubes, not shown, which lead to the outlets 252. In this way any water which might enter through the openings 312 cannot reach an interior of the base but, instead, is directed to the outlets 252.

A safety switch 290 is mounted to the platform 270. Unless the switch 290 is closed the apparatus cannot be rendered operably by means of the switch 222.

The UV source 284 preferably comprises a number of light emitting diodes 284A, 284B . . . 284N which are placed at spaced apart locations on the elongate tubular member 216. Alternatively or additionally the UV source 284 includes a source 284X of an appropriate shape which is mounted to the platform 270.

FIG. 11 shows a housing 320 which is positioned over the container 260 thereby to enclose the container and the UV source 284. The housing 320 is made from a material which is opaque to UV radiation. Thus, when the housing is in place and the UV source is operable, a person adjacent the apparatus 210 will not be exposed to and cannot see the UV radiation. In this way possible tissue or eye damage to a user is avoided.

In use of the apparatus 210 the container 260 is washed by a person in an appropriate manner. After the container has been rinsed it is placed, inverted as shown in FIG. 11, over the elongate tubular member 216 with the mouth 268 resting on the platform. The housing 320 is positioned as shown. The safety switch 290 is thereby closed. A user can then activate the apparatus 210 by means of the switch 222, The heater coil 280 is then energised by means of power from the batteries 293 and the fan 282 is simultaneously activated. The ultraviolet source 284 is also energised.

Heated and dry air is forced into the elongate tubular member 216 and exits from the bore 232 via the slots 240 into an interior of the container. The heated and dry air impinges on an inner surface 262A of the wall 262 which dries and forces moisture on the surface 262A through the mouth 268. The volume is air-pressurised to some extent but air does escape through the mouth 268 and then leaves the housing through the corresponding outlets 252. If appropriate, the housing 320 can be similar to the housing 40 in FIG. 1.

The heating, drying and radiating process continues for a period determined by the timer 278. This can be set by a user according to requirements. At the end of this period the controller 276 causes a signal to be displayed on the display 226. At that time the fan, the heater coil and the UV source are no longer energised.

At any time if the housing 320 is removed the safety switch 290 automatically interrupts operation of at least the UV source.

The apparatus could include a number of the elongate tubular members 216 which are of different sizes and which are interchangeable so that the apparatus can be adapted for use easily with a variety of objects, not necessarily food containers, of different sizes and shapes—e.g. as is shown in FIGS. 8 and 9.

Features of the apparatus 10 can, as appropriate be included in the apparatus 210, and vice versa.

1-14. (canceled)

15. An apparatus for treating an object, the apparatus comprising a base in which a passage is located, a vent inlet to the passage, an outlet from the passage, the base including a platform at which said outlet from the passage is positioned, a housing which is impervious to ultraviolet radiation and which includes a volume within which the at least one object is removably located, the housing being configured to be engaged with the platform whereby the outlet from the passage is placed in air communication with the volume, an air heater, an air fan and a controller for regulating operation of the air heater and the fan thereby to produce a stream of air which is at a predetermined air temperature and which is directed through the passage and from the outlet into the volume, an ultraviolet source positioned on the platform to direct ultraviolet radiation into the volume, and a water outlet from the passage which is located at what in use is a lowermost point of the passage.

16. The apparatus according to claim 15 wherein said controller is operable to produce said stream of conditioned air at a predetermined air flow rate.

17. The apparatus according to claim 15 wherein, in use, said vent inlet to the passage is higher than said air fan and said air heater.

18. The apparatus according to claim 15 wherein the ultraviolet source, which is operable by the controller, is located at said outlet from the passage.

19. The apparatus according to claim 15 which includes a safety switch at the platform which prevents operation at least of the ultraviolet source unless the housing is engaged with the platform.

20. The apparatus according to claim 15 which includes a support which is inside said volume in the housing and which is configured to support at least said object inside said volume.

21. The apparatus according to claim 15 wherein the platform is configured to support a container which is inside said volume and which has a mouth which is positioned over said outlet from the passage.

22. The apparatus according to claim 15 which includes an air escape port from the housing.

23. The apparatus according to claim 16 which includes a fuse which, in response to a temperature of said stream of conditioned air above a threshold value, inhibits further operation at least of the air heater.

24. The apparatus according to claim 21 which includes an elongate tubular member which is configured to extend from the platform through the mouth into an interior of the container to allow the stream of air to be emitted into the interior of the container through outlets from the tubular member.
